(19) United States
(12) Patent Application Publication
(10) Pub. No.: US 2021/0378995 A1
(43) Pub. Date: Dec. 9, 2021

(54) METHODS AND TOPICAL PHARMACEUTICAL COMPOSITIONS FOR THE TREATMENT OF SKIN MICROVASCULAR DYSFUNCTION

(71) Applicants: INSERM (INSTITUT NATIONAL DE LA SANTÉ ET DE LA RECHERCHE MÉDICALE), Paris (FR); UNIVERSITÉ DE ROUEN NORMANDIE, Mont-Saint-Aignan (FR); CENTRE HOSPITALIER UNIVERSITAIRE DE ROUEN, Rouen (FR); UNIVERSITÉ GRENOBLE ALPES, Saint Martin d'Heres (FR); CENTRE HOSPITALIER UNIVERSITAIRE DE GRENOBLE, La Tranche (FR)

(72) Inventors: Jérémy BELLIEN, Rouen cedex (FR); Frédéric BOUNOURE, Rouen cedex (FR); Mohamed SKIBA, Rouen cedex (FR); Yann SAVINA, La Tronche (FR); Matthieu ROUSTIT, La Tronche (FR); Jean-Luc CRACOWSKI, La Tronche (FR)

(21) Appl. No.: 17/282,428

(22) PCT Filed: Oct. 9, 2019
(86) PCT No.: PCT/EP2019/077282

§ 371 Date: Apr. 2, 2021

(30) Foreign Application Priority Data
Oct. 10, 2018 (EP) 18306336.1

(51) Int. Cl. A61K 31/17; A61K 31/197; A61K 31/4468; A61K 31/53; A61P 17/00; A61P 9/14; A61P 3/10

(52) U.S. Cl. CPC A61K 31/17; A61K 31/197; A61K 31/4468; A61P 3/10; A61P 17/00; A61P 9/14; A61K 31/53

(57) ABSTRACT

Microvascular dysfunction remains a major contributor to the development of skin complications. The inventors assessed the impact of the local inhibition of soluble epoxide hydrolase (sEH), which metabolizes vasodilator and anti-inflammatory epoxyeicosanoids, on the diabetic skin microvascular dysfunction. The inventors have therefore developed some formulations of sEH inhibitors (GSK2256294 and t-AUCB) for topical administration. In particular, they show that an aqueous gel containing 400 mg/L t-AUCB dissolved in 50% dimethy lsulfo xide (DMSO) allowed a stable and continuous diffusion of t-AUCB from 2 hours after application on skin pig ears to over a period of 24 h. Compared to a control gel, the gel with t-AUCB did not significantly modify the basal skin blood flow but improved the altered hyperemic response of db/db mice 2 hours after application. The results show that the topical administration of a sEH inhibitor improves the skin microcirculatory function, representing a promising pharmacological approach to prevent the development of skin complications especially in diabetic patients.

### FIELD OF THE INVENTION

The present invention relates to methods and topical pharmaceutical compositions for the treatment of skin microvascular dysfunction.

### BACKGROUND OF THE INVENTION

Skin microvascular dysfunction referring to abnormalities in the structure and/or function of small blood vessels in the skin is hallmark of several diseases and conditions. For instance, diabetes, aging and high blood pressure due to e.g. extended bed rest can impair microvascular circulation and lead to changes in the skin on the lower extremities, which in turn, can lead to formation of ulcers and subsequent infection. Microvascular changes lead to limb muscle microangiopathy, as well as a predisposition to develop peripheral ischemia and a reduced angiogenesis compensatory response to ischemic events. Foot ulcers and gangrene are frequent comorbid conditions of peripheral arterial disease (PAD). Concurrent peripheral neuropathy with impaired sensation make the foot susceptible to trauma, ulceration, and infection. Diabetic foot ulcers may occur not only in conjunction with PAD but may also be associated with neuropathy, venous insufficiency (varicose veins), trauma, and infection. Accordingly, it would be beneficial to have a pharmaceutical compositions that could better treat skin microvascular dysfunction.

### SUMMARY OF THE INVENTION

The present invention relates to methods and topical pharmaceutical compositions for the treatment of skin microvascular dysfunction. In particular, the present invention is defined by the claims.

### DETAILED DESCRIPTION OF THE INVENTION

Microvascular dysfunction remains a major contributor to the development of skin complications. The inventors assessed the impact of the local inhibition of soluble epoxide hydrolase (sEH), which metabolizes vasodilator and anti-inflammatory epoxyeicosanoids, on the diabetic skin microvascular dysfunction. The inventors have therefore developed some formulations of sEH inhibitors (GSK2256294 and t-AUCB) for topical administration. In particular, they show that an aqueous gel containing 400 mg/L t-AUCB dissolved in 50% dimethylsulfoxide (DMSO) allowed a stable and continuous diffusion of t-AUCB from 2 hours after application on skin pig ears to over a period of 24 h. In the thinner dorsal skin of db/db mice, a maximal concentration of t-AUCB was detected 2 hours after gel application and quickly decreased thereafter. Systemic diffusion of t-AUCB appeared limited with a plasma levels of t-AUCB above the mouse sEH IC50 in 17% of the treated animals. Compared to a control gel only containing 50% DMSO, the gel with t-AUCB did not significantly modify the basal skin blood flow but improved the altered hyperemic response of db/db mice 2 hours after application. HES histological staining demonstrated that skin integrity was preserved. These results show that the topical administration of a sEH inhibitor improves the skin microcirculatory function, representing a promising pharmacological approach to prevent the development of skin complications especially in diabetic patients.

Accordingly, the present invention relates to a method of treating skin microvascular dysfunction in a subject in need thereof comprising topically administering the subject with a therapeutically effective amount of a sEH inhibitor.

As used herein, the term “skin microvascular dysfunction” has its general meaning in the art and refers to the abnormalities in the structure and/or function of small blood vessels, such as arterioles, capillaries and venules present in the skin. Microvascular dysfunction can be assessed and determined by analyzing vascular permeability, electromicroscopy of microvasculature, and analysis of the levels of endothelial and pericyte markers (such as CD-31, isolectin B4, PDGFRβ and NG2), among others.

In some embodiments, the subject suffers from diabetes mellitus. As used herein, the term “diabetes mellitus” refers to a disease caused by a relative or absolute lack of insulin leading to uncontrolled carbohydrate metabolism, commonly simplified to “diabetes,” though diabetes mellitus should not be confused with diabetes insipidus. As used herein, “diabetes” refers to diabetes mellitus, unless otherwise indicated. A “diabetic condition” includes prediabetes and diabetes. Type 1 diabetes (sometimes referred to as “insulin-dependent diabetes” or “juvenile-onset diabetes”) is an auto-immune disease characterized by destruction of the pancreatic β cells that leads to a total or near total lack of insulin. In type 2 diabetes (T2DM; sometimes referred to as “non-insulin-dependent diabetes” or “adult-onset diabetes”), the body does not respond to insulin, though it is present. Thus the method of the present invention is particularly suitable for the treatment of diabetic dermopathy. Moreover the method of the present invention is also particularly suitable for the treatment of diabetic ulcers, in particular diabetic foot ulcers. As used herein, the term “diabetic ulcer” refers to ulcerations, including foot ulcerations, due to microvascular dysfunction associated with diabetes.

In some embodiments, the subject suffers from systemic sclerosis (SSc). As used herein the term “systemic sclerosis” has its general meaning in the art and refers to an auto-immune disorder characterized by vascular alterations and fibrosis of the skin that results in particular from microvascular dysfunction.

In some embodiments, the subject suffers from a disease or condition selected from the group consisting of inherited or recessive myopathies (such as muscular dystrophies), muscle-wasting diseases (such as cachexia that may be the result from underlying illnesses such as acquired immunodeficiency diseases [AIDS], rheumatoid arthritis, cancer, chronic obstructive pulmonary disease [COPD], and cirrhosis), conditions of muscle atrophy or attenuation (such as sarcopenia that may be the result of aging), protracted disuse (such as paralysis, coma, extended bed rest, and Intensive Care Unit (ICU) stay), weakness induced by surgery (such as joint replacement surgery), drug-induced myopathy and rhabdomyolysis.

In some embodiments, the method of the present invention is particularly suitable for the treatment of wound. As used herein, the term “wound” denotes a bodily injury with disruption of the normal integrity of tissue structures. The term is also intended to encompass the terms “sore,” “lesion,” “necrosis,” and “ulcer.” Normally, the term “sore” is a popular term for almost any lesion of the skin or mucous membranes and the term “ulcer” is a local defect, or excavation, of the surface of an organ or tissue, which is produced by the sloughing of necrotic tissue. Lesion generally relates to any tissue defect. Necrosis is related to dead tissue resulting from infection, injury, inflammation or infarctions.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “soluble epoxide hydrolase” or “sEH” has its general meaning in the art and refers to an epoxide hydrolase which in many cell types converts epoxyeicosatrienoic acids (EETs) to dihydroxy derivatives called dihydroxyeicosatrienoic acids (“DHETs”). The cloning, sequence, and accession numbers of the human sEH sequence are set forth in Beetham et al., Arch. Biochem. Biophys. 305(1):197-201 (1993).

As used herein, the term “sEH inhibitor” or “soluble epoxide hydrolase” refer to a compound that is capable of inhibiting the hydrolase activity of sEH. sEH inhibitors are well known in the art. A variety of chemical structures have been described for sEH inhibitors. Derivatives in which the urea, carbamate or amide pharmacophore are particularly useful as sEH inhibitors. For instance selective and competitive inhibition of sEH in vitro by a variety of urea, carbamate, and amide derivatives is taught, for example, by Morisseau et al., Proc. Natl. Acad. Sci. U.S.A, 96:8849-8854 (1999), which provides substantial guidance on designing urea derivatives that inhibit the enzyme. Various disclosures of sEH inhibitors also include:
Ingraham R H, Gless R D, Lo H Y. Soluble epoxide hydrolase inhibitors and their potential for treatment of multiple pathologic conditions. Curr Med Chem. 2011; 18(4):587-603. Review. PubMed PMID: 21143109.
Qiu H, Li N, Liu J Y, Harris T R, Hammock B D, Chiamvimonvat N. Soluble epoxide hydrolase inhibitors and heart failure. Cardiovasc Ther. 2011 April; 29(2):99-111.
Marino J P Jr. Soluble epoxide hydrolase, a target with multiple opportunities for cardiovascular drug discovery. Curr Top Med Chem. 2009; 9(5):452-63. Review.
Shen H C. Soluble epoxide hydrolase inhibitors: a patent review. Expert Opin Ther Pat. 2010 July; 20(7):941-56. doi: 10.1517/13543776.2010.484804. Review.
Morisseau C., Hammock D. B., Impact of Soluble Epoxide Hydrolase and Epoxyeicosanoids on Human Health. Annu Rev Pharmacol Toxicol. 2013; 53:37-58. doi: 10.1146/annurev-pharmtox-011112-140244.

A number of other sEH inhibitors which can be used in tin the method of the present invention include PCT/US2012/025074, PCT/US2011/064474, PCT/US2011/022901, PCT/US2008/072199, PCT/US2007/006412, PCT/US2005/038282, PCT/US2005/08765, PCT/US2004/010298 and U.S. Published Patent Application Publication 2005/0026844, each of which is hereby incorporated herein by reference in its entirety for all purposes. U.S. Pat. No. 5,955,496 also sets forth a number of sEH inhibitors which can be used in the methods. Additional inhibitors of sEH suitable for use in the methods are set forth in U.S. Pat. Nos. 6,150,415 and 6,531,506.

In some embodiments, the inhibitor of sEH is selected from the group consisting of 3-(4-chlorophenyl)-1-(3,4-dichlorphenyl)urea or 3,4,4′-trichlorocarbanilide (TCC); 12-(3-adamantan-1-yl-ureido) dodecanoic acid (AUDA); 1-adamantanyl-3-{5-[2-(2-ethoxyethoxy)ethoxy]pentyl]}urea (AEPU); 1-(1-acetypiperidin-4-yl)-3-adamantanylurea (APAU); trans-4-[4-(3-Adamantan-1-yl-ureido)-cyclohexyloxy]-benzoic acid (tAUCB); cis-4-[4-(3-Adamantan-1-yl-ureido)-cyclohexyloxy]-benzoic acid (cAUCB); 1-(1-methylsulfonyl-piperidin-4-yl)-3-(4-trifluoromethoxy-phenyl)-urea (TUPS); trans-4-{4-[3-(4-Trifluoromethoxy-phenyl)-ureido]-cyclohexyloxy}-benzoic acid (tTUCB); 1-trifluoromethoxyphenyl-3-(1-propionylpiperidin-4-yl) urea (TPPU); 1-(1-ethylsulfonyl-piperidin-4-yl)-3-(4-trifluoromethoxy-phenyl)-urea (TUPSE) 1-(1-(cyclopropanecarbonyl)piperidin-4-yl)-3-(4-(trifluoromethoxy)phenyl)urea (CPTU); trans-N-methyl-4-[4-(3-Adamantan-1-yl-ureido)-cyclohexyloxy]-benzamide (tMAUCB;) trans-N-methyl-4-[4-((3-trifluoromethyl-4-chlorophenyl)-ureido)-cyclohexyloxy]-benzamide (tMTCUCB); cis-N-methyl-4-{4-[3-(4-trifluoromethoxy-phenyl)-ureido]-cyclohexyloxy}-benzamide (cMTUCB); and 1-cycloheptyl-3-(3-(1,5-diphenyl-1H-pyrazol-3-yl)propyl)urea (HDP3U).

In some embodiments, the sEH inhibitor of the present invention is GSK2256294A ((1R,3S)—N-[[4-cyano-2-(trifluoromethyl)phenyl]methyl]-3-[[4-methyl-6-(methylamino)-1,3,5-triazin-2-yl]amino]cyclohexane-1-carboxamide), which has the formula of

In some embodiments, the sEH inhibitor of the present invention is tAUCB (trans-4-[4-(3-Adamantan-1-yl-ureido)-cyclohexyloxy]-benzoic acid), which has the formula of:

In some embodiments, the inhibitor of sEH of the present invention is Sorafenib (4-[4-({[4-chloro-3-(trifluoromethyl)phenyl]carbamoyl}amino)phenoxy]-N-methylpyridine-2 carboxamide), which has the formula of:

As used herein, the term “topical administration” is used herein in its conventional sense to mean delivery of a pharmacologically active agent (i.e. the sEH inhibitor) to the skin.

By a “therapeutically effective amount” is meant a sufficient amount of the sEH inhibitor to treat microvascular dysfunction at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the agent for the symptomatic adjustment of the dosage to the subject to be treated. A medicament typically contains from about 0.01 mg to about 500 mg of the agent, preferably from 1 mg to about 100 mg of the agent. An effective amount of the drug is ordinarily supplied at a dosage level from 0.0002 mg/kg to about 20 mg/kg of body weight per day, especially from about 0.001 mg/kg to 7 mg/kg of body weight per day.

According to the present invention the sEH inhibitor of the present invention is administered to the subject via a topical formulation. As used herein the term “topical formulation” refers to a formulation that may be applied to skin. Topical formulations can be used for both topical and transdermal administration of substances. As used herein, “topical administration” is used in its conventional sense to mean delivery of a substance, such as a therapeutically active agent, to the skin or a localized region of a subject's body. As used herein, “transdermal administration” refers to administration through the skin. Transdermal administration is often applied where systemic delivery of an active is desired, although it may also be useful for delivering an active to tissues underlying the skin with minimal systemic absorption. Typically, the topical pharmaceutically acceptable carrier is any substantially nontoxic carrier conventionally usable for topical administration of pharmaceuticals in which the sEH inhibitor of the present invention will remain stable and bioavailable when applied directly to skin surfaces. For example, carriers such as those known in the art effective for penetrating the keratin layer of the skin into the stratum comeum may be useful in delivering the sEH inhibitor of the present invention to the area of interest. Such carriers include liposomes. sEH inhibitor of the present invention can be dispersed or emulsified in a medium in a conventional manner to form a liquid preparation or mixed with a semi-solid (gel) or solid carrier to form a paste, powder, ointment, cream, lotion or the like. Suitable topical pharmaceutically acceptable carriers include water, buffered saline, petroleum jelly (vaseline), petrolatum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, synthetic polymers, alcohols, polyols, and the like. The carrier can be a water miscible carrier composition. Such water miscible, topical pharmaceutically acceptable carrier composition can include those made with one or more appropriate ingredients outset of therapy. The topical acceptable carrier will be any substantially non-toxic carrier conventionally usable for topical administration in which sEH inhibitor of the present invention will remain stable and bioavailable when applied directly to the skin surface. Suitable cosmetically acceptable carriers are known to those of skill in the art and include, but are not limited to, cosmetically acceptable liquids, creams, oils, lotions, ointments, gels, or solids, such as conventional cosmetic night creams, foundation creams, suntan lotions, sunscreens, hand lotions, make-up and make-up bases, masks and the like. Any suitable carrier or vehicle effective for topical administration to a patient as known in the art may be used, such as, for example, a cream base, creams, liniments, gels, lotions, ointments, foams, solutions, suspensions, emulsions, pastes, aqueous mixtures, sprays, aerosolized mixtures, oils such as Crisco®, soft-soap, as well as any other preparation that is pharmaceutically suitable for topical administration on human and/or animal body surfaces such as skin or mucous membranes. Topical acceptable carriers may be similar or identical in nature to the above described topical pharmaceutically acceptable carriers. It may be desirable to have a delivery system that controls the release of sEH inhibitor of the present invention to the skin and adheres to or maintains itself on the skin for an extended period of time to increase the contact time of the sEH inhibitor of the present invention on the skin. Sustained or delayed release of sEH inhibitor of the present invention provides a more efficient administration resulting in less frequent and/or decreased dosage of sEH inhibitor of the present invention and better patient compliance. Examples of suitable carriers for sustained or delayed release in a moist environment include gelatin, gum arabic, xanthane polymers. Pharmaceutical carriers capable of releasing the sEH inhibitor of the present invention when exposed to any oily, fatty, waxy, or moist environment on the area being treated, include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like. Controlled delivery systems are described, for example, in U.S. Pat. No. 5,427,778 which provides gel formulations and viscous solutions for delivery of the sEH inhibitor of the present invention to a skin site. Gels have the advantages of having a high water content to keep the skin moist, the ability to absorb skin exudate, easy application and easy removal by washing. Preferably, the sustained or delayed release carrier is a gel, liposome, microsponge or microsphere. The sEH inhibitor of the present invention can also be administered in combination with other pharmaceutically effective agents including, but not limited to, antibiotics, other skin healing agents, and antioxidants. In some embodiments, the topical formulation of the present invention comprises a penetration enhancer. As used herein, “penetration enhancer” refers to an agent that improves the transport of molecules such as an active agent (e.g., a drug) into or through the skin. Various conditions may occur at different sites in the body either in the skin or below creating a need to target delivery of compounds. Thus, a “penetration enhancer” may be used to assist in the delivery of an active agent directly to the skin or underlying tissue or indirectly to the site of the disease or a symptom thereof through systemic distribution. A penetration enhancer may be a pure substance or may comprise a mixture of different chemical entities.

The invention will be further illustrated by the following figures and examples. However, these examples and figures should not be interpreted in any way as limiting the scope of the present invention.

### FIGURES

FIG. 1: Influence of the solvent on the percutaneous absorption of t-AUCB using Franz's cells during 24 hours on pigskin (n=3)

FIG. 2: Influence of the solvent on the percutaneous absorption of GSK2256294 using Franz's cells during 24 hours on pigskin (n=3)

FIG. 3: Percutaneous passage of t-AUCB using in Franz's cells during 24 hours on pigskin with solutions DMSO/water (n=3)

FIG. 4: Percutaneous passage of t-AUCB using Franz's cells during 24 hours on pigskin with hydroalcoholic solutions (n=3)

FIG. 5. Basal skin blood flow (A) and thermal hyperemia (B) measured by laser Doppler imaging in control (n=7) and db/db mice (n=32). A.P.U.: arbitrary perfusion unit.

FIG. 6. Evolution of basal skin blood flow (A) and thermal hyperemia (B) measured by laser Doppler imaging after a 2-hour topical application of the t-AUCB-containing gel and the vehicle control gel on the dorsal skin of db/db mice (n=13). *P<0.05 vs. before topical application, ^{†}P<0.05 vs. vehicle control gel. A.P.U.: arbitrary perfusion unit.

FIG. 7. A, Skin levels of t-AUCB, quantified by liquid chromatography coupled to tandem mass spectrometry, 2 and 24 hours after the topical application of the t-AUCB-containing gel on the dorsal skin of db/db mice (n=6 per time point). B, Thermal hyperemia measured by laser Doppler imaging before, 2 and 24 hours after the topical application of the t-AUCB-containing gel on the dorsal skin of db/db mice (n=6-26 per time point). *P<0.05 vs. before topical application. A.P.U.: arbitrary perfusion unit.

FIG. 8. Evolution of t-AUCB level, quantified by liquid chromatography coupled to tandem mass spectrometry, in the receptor compartment of Franz cells from 0 to 24 hours after gel application on pig ear skin (n=3 per time point).

FIGS. 9. (A-B) Evolution of GSK2256294 level, quantified by liquid chromatography coupled to tandem mass spectrometry, in the receptor compartment of Franz cells from 0 to 24 hours after topical application on pig ear skin (triplicate).

### EXAMPLES

### Example 1: Preformulation

Method:

The t-AUCB and GSK2256294 percutaneous absorptions were determined using Franz's cell. The skin of a pig's ears was chosen for the experiments as it is very similar to that of a human skin. Franz's cells had a contact area of 2 cm² and the experiments were conducted at 32° C. The donor compartment was filled by a solution of 2 mL at 4 μg/g of t-AUCB and 2 mL at 40 μg/mL, 20 μg/mL or 4 μg/mL of GSK2256294. Various vehicles were tested to determine the most favourable one to the cutaneous absorption. The receptor compartment contained 4.5 mL of PBS and was under magnetic stirring. Samples from the receptor compartment were collected for 24 hours at different times to determine the flow of t-AUCB or GSK2256294 percutaneous absorption. Samples were frozen at −20° C. The t-AUCB or GSK2256294 quantification was realized by HPLC/MS/MSS.

Results:

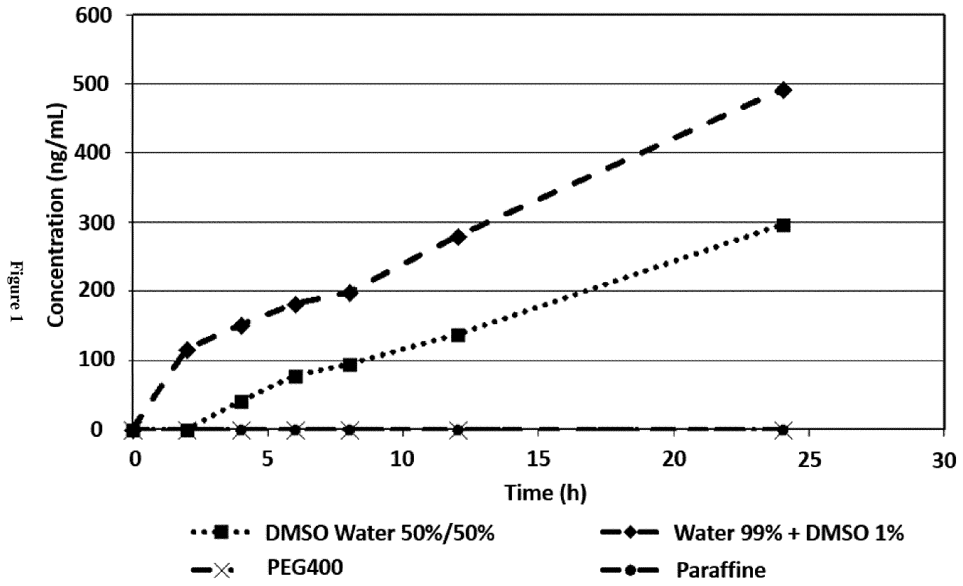

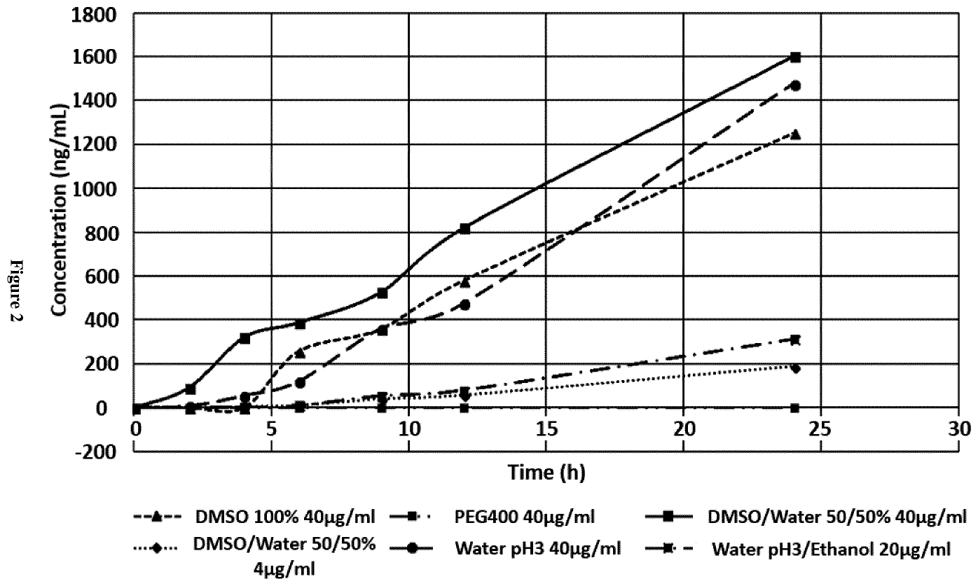

The percutaneous absorption study was conducted with a solution of t-AUCB at 4 μg/g in 4 different vehicles: PEG 400, Dimethylsulfoxide (DMSO) 50%/Water 50%, Water 99%/DMSO 1% and paraffin wax. The vehicle effect is represented in FIG. 1. A t-AUCB percutaneous absorption was noticed only with DMSO/water using a 50%/50% ratio and with water/DMSO 99%/1%. The flow was 26.7 ng/cm²/h with DMSO/water to 50%/50% and 38 ng/cm²/h with water/DMSO 99%/1%. No flow was found with PEG400 and paraffin wax.

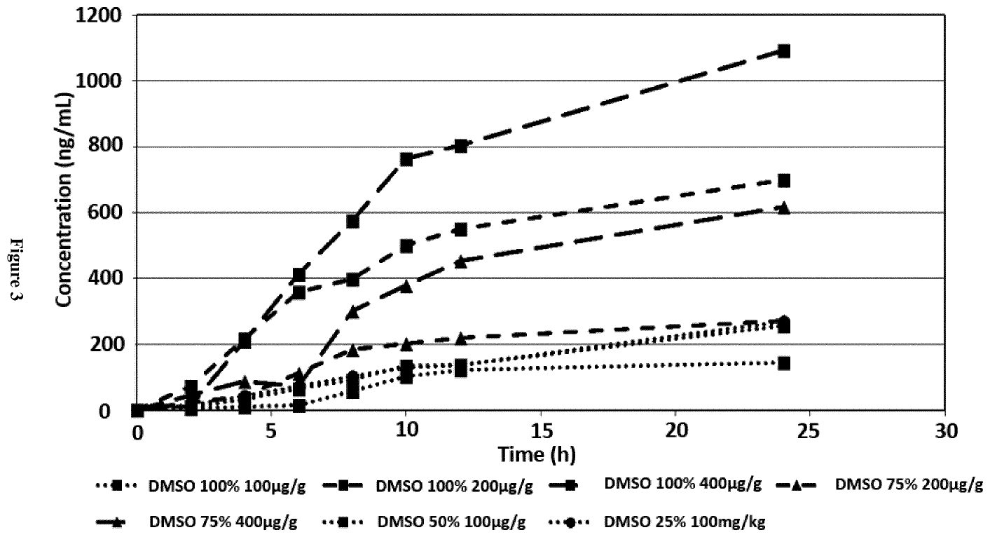

The GSK2256294 percutaneous absorption study was done using different vehicles: PEG 400, DMSO/Water 50%/50%, Water at a pH of 3, Water at pH3/alcohol 50/50%. Paraffin wax could not be used because of the lack of solubility of GSK2256294. The vehicle effect is represented in FIG. 2. A GSK2256294 percutaneous absorption was noticed with DMSO 50%/Water 50%, Water at a pH of 3 and Water at pH3/alcohol 50/50%. No flow was found with PEG400. The flow was 167.4 ng/cm²/h with DMSO/water to 50%/50% at 40 μg/mL and 21.9 ng/cm²/h at 4 μg/mL. 9.02% and 10.5% of GSK2256294 were absorbed and released by the pigskin over 24 hrs. With DMSO, the flow was 134.1 ng/cm²/h at 40 μg/mL with a passage rate of 7.04%. The flows with water at pH3 and alcohol at 50% were respectively 167.4 ng/cm²/h and 38.6 ng/cm²/h with a passage rate of 8.31% and 3.52%.

Because of t-AUCB and GSK2256294 lipophilic properties, the use of a polar vehicle is more favourable to its transcutaneous passage than unpolar one.

### Example 2: Formulations Using DMSO

Method:

The t-AUCB topical formulation used DMSO and water as vehicle. The experiments were conducted with Franz's cell using the skin of a pig's ear. The donor compartment was filled by 13 μL of DMSO solution with a concentration between 100 to 400 μg/g of t-AUCB. The receptor medium was constituted by 4.5 mL of PBS and was at 32° C. and under magnetic stirring. Samples from the receptor compartment were collected at different times over 24 hours to determine the passage flow of t-AUCB. Samples were then frozen at −20° C. The t-AUCB quantification was realized by HPLC/MS/MSS.

Results:

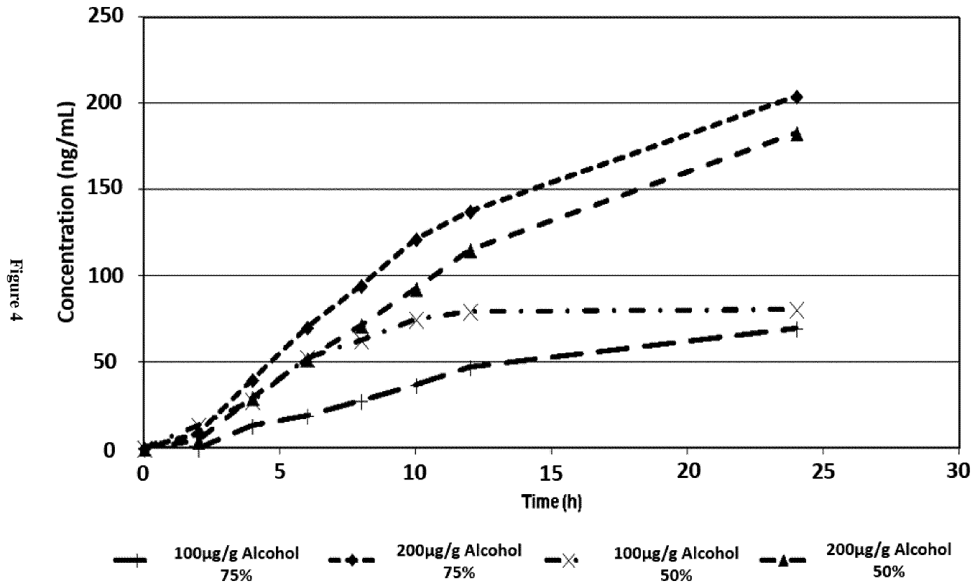

The t-AUCB flows with a concentration between 100 to 400 μg/g in DMSO solution between 25% and 100% are represented in FIG. 3. The most important flow is obtained with the solution of t-AUCB with a concentration of 400 μg/g and in the 100% DMSO with 131.26 ng/cm²/h. The solution containing 25% of DMSO and 100 μg/g of t-AUCB presented a flow of 19.59 ng/cm²/h. The passage rate after 24 h is between 110.6% for DMSO 100% 200 μg/g and 22.4% for DMSO 25% 100 μg/g. A slowing down of the passage t-AUCB was also noticed after 10 hours in all samples. This seems to indicate the necessity of an application twice a day. The lag time is evaluated to 2 h.

### Example 3: Formulations Using Alcohol

Method:

Alcohol and water were used as a vehicle for t-AUCB's topical formulation. The experiments were conducted with Franz's cell using the skin of a pig's ear. The donor compartment was filled by an 13 μL of alcoholic solution with a concentration between 100 to 200 μg/g of t-AUCB The receptor medium was constituted by 4.5 mL of PBS, was at 32° C. and under magnetic stirring. Samples from the receptor compartment were collected at different times over 24 hours to determine the passage flow of t-AUCB. Samples were then frozen at −20° C. The t-AUCB quantification was realized by HPLC/MS/MSS.

Results:

FIG. 4 represents the passage of hydroalcoholic solutions of t-AUCB with an alcohol content between 50 and 75% and with a t-AUCB concentration between 100 and 200 μg/g. The percutaneous flow is higher with either a larger alcohol content or a larger concentration of t-AUCB. The most important flow was obtained by a t-AUCB solution 200 μg/g in alcohol 75% with a 24.63 ng/cm²/h and with a passage rate of 35.3%. The flow and passage rate were 23.2 ng/cm²/h and 31.7% with t-AUCB solution 200 μg/g in alcohol 50% and 9.78 ng/cm²/h and 27.8% with t-AUCB solution 100 μg/g in alcohol 50%. The flow through the pigskin was effective from 4 h to 10 h.

### Example 4: Impact of the Local Inhibition of Soluble Epoxide Hydrolase on Diabetic Skin Microcirculatory Dysfunction

Methods:

Animals and Treatments

The protocol was approved by a local institutional review committee (agreement number C 38 516 10 006, n^{o}2017011312598602-V5#8531) and conducted in accordance with the National Institutes of Health (NIH) Guide for the Care and Use of Laboratory Animals. Nine-week-old male wild-type C57BL/6J and db/db (BKS(D)-Leprdb/JOrlRj) mice, a genetic model of type 2 diabetes, were acquired from Janvier Labs (Le Genest-Saint-Isle, France). These mice were allowed to acclimate to the photoperiod (12-hour light/12-hour dark) and temperature conditions (22±1° C.) for one week prior to the start of the study. A 2-hour topical administration (20 μL) of a newly developed gel-like, aqueous pharmaceutical preparation containing the sEH inhibitor trans-4-(4-(3-adamantan-1-yl-ureido)-cyclohexyloxy)-benzoic acid (t-AUCB: 400 mg/L) dissolved in dimethyl sulfoxide (DMSO) or a vehicle control gel was performed on the dorsal skin of db/db mice, depilated two days before experiments. Assessment of microvascular function, skin biopsies (50 mm²) and intra-cardiac blood sampling were performed 2 and 24 hours after gel application. Animals were anaesthetized with isoflurane (induction at 3% during 3 minutes, and then maintained at 2%) and placed over a heating carpet to maintain stable core temperature (37.5±0.5° C.).

Local and Systemic Quantification of t-AUCB

Plasma and skin levels of t-AUCB were quantified by liquid chromatography coupled to tandem mass spectrometry (LC-MS/MS) (13). Briefly, skin tissues were mixed with 1 mL of methanol-water (50:50 v/v) and ultrasonicated for 10 min, or 100 μL of plasma were mixed with 300 μL of methanol, allowing protein precipitation. Then, skin and plasma samples were thoroughly vortexed for 10 see and centrifuged at 16,100 g for 5 min. The resulting supernatants were collected and analyzed by LC-MS/MS. Chromatographic separation was performed on a Kinetex C18 column (2.6-μm particle size, 50-mm length×3-mm inner diameter). The autosampler temperature was set at 8° C., the column oven at 30° C., the injected volume was 20 μL and the flow rate was 400 μL/min. The mobile phase was 0.2% formic acid in methanol (solvent A) and 2 mM ammonium formate with 0.2% formic acid in water (solvent B). The elution started with 95% B (0-2 min), 95-5% B (2-5 min), 5% B (5-10 min), 5-95% B (10-11 min), 95% B (11-12 min). The following multiple reaction monitoring (MRM) transitions m/z 412.9 to m/z 135.1 and m/z 412.9 to m/z 93.0 in positive ion mode were used to detect t-AUCB (quantification and confirmation transitions respectively). Skin levels were normalized to tissue weight.

Assessment of Skin Microvascular Function

Skin microvascular reactivity to local heating was used as an index of endothelium-dependent function (14). Dorsal skin blood flow was measured by laser Doppler imaging (LDI; PeriScan PIM, Perimed, Jarfilla, Sweden) over 10 minutes before heating (baseline flow). The skin was subsequently heated at 41° C. during 20 minutes using a 0.5 cm² heating probe regulated with an internal thermometer. Skin blood flow was then recorded during the following 15 minutes.

Data were digitized, stored on a computer, and analysed offline with signal processing software (PimSoft v1.5.4.8078, Perimed, Järfälla, Sweden). Baseline and peak hyperaemia were expressed as arbitrary perfusion units (APUs), averaged over 3 minutes immediately before, and 1 minute immediately after heating, respectively. Thermal hyperemia was subsequently calculated as the difference between peak hyperaemia and baseline skin blood flow.

Skin Integrity

Skin biopsies were carefully sampled and immediately fixed in a 4% formalin solution for 24 hours. After proper fixation, tissue samples were embedded in paraffin and stored at room temperature until analysis. 4 m sections were deparaffinized and stained with standard Hematoxylin-Eosin (H&E) staining. Slides were analyzed by an experienced pathologist (JMP).

Transdermal Passage of t-AUCB Across Pig Ear Skin

The transdermal passage of t-AUCB across pig ear skin, which is structurally closer to human skin than mouse skin, was assessed using Franz diffusion cells as previously described (15).

Statistical Analysis

All values are expressed as mean±SEM. The Shapiro-Wilk test was used to assess normality. Analyses of the differences between diabetic and control mice for basal skin blood flow and thermal hyperemia were performed by unpaired t-test or nonparametric Mann-Whitney rank-sum test. Analyses of the variation in basal skin blood flow and thermal hyperemia induced by the t-AUCB-containing gel were performed using mixed effects models with time as fixed effect and mouse as a random effect followed in case of significance by Bonferroni post-hoc tests to compare the value obtained before application to other time points after application. Analyses of the differences between the impact of the t-AUCB-containing gel and the vehicle gel on basal skin blood flow and thermal hyperemia were performed by repeated-measures ANOVA, and we assessed the effect of the group, of time and the time*group interaction. Statistical analysis was performed with NCSS software (version 07.1.14). A two-sided P<0.05 was considered statistically significant.

Results:

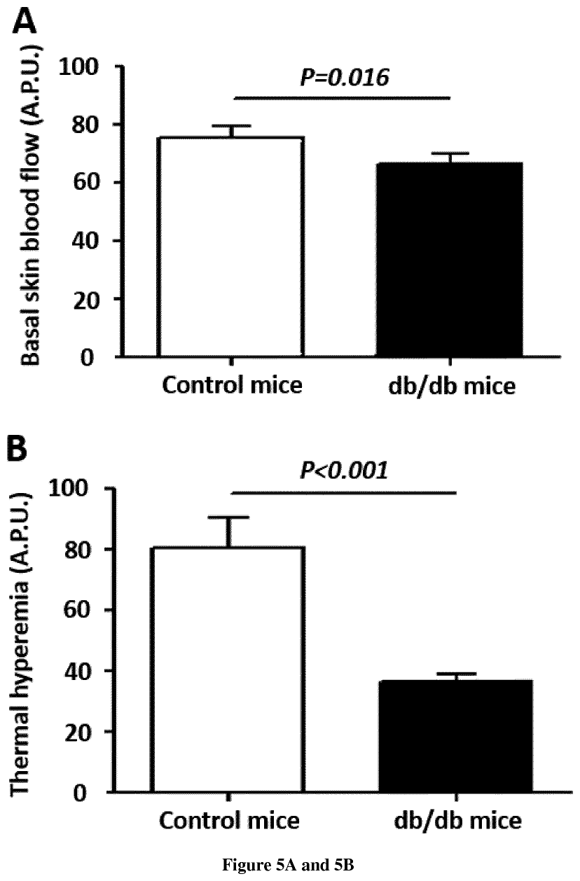

At untreated skin sites, cutaneous blood flow was slightly but significantly lower in db/db mice compared to control mice (FIG. 5A). In contrast, there was a marked reduction in thermal hyperemia in db/db mice compared to controls (FIG. 5B), demonstrating the presence of diabetic skin microcirculatory dysfunction.

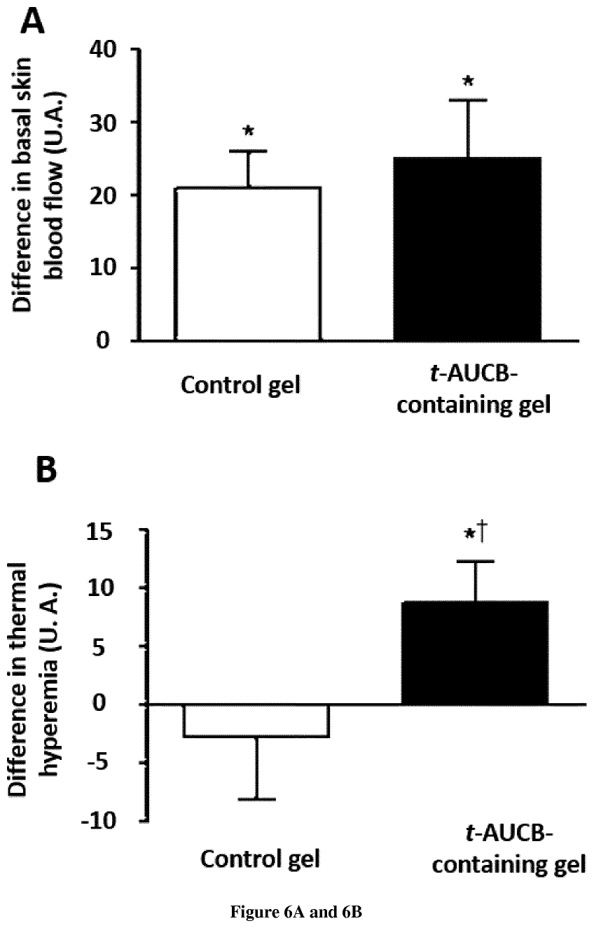

We carefully compared the impact of the t-AUCB-containing gel to a vehicle control gel in db/db mice. Both gels were applied on the same animal, with a minimal distance of 1 cm between the two application sites. Both gels increased basal skin blood flow after a 2-hour application, with no significant difference between groups (FIG. 6A). However, the t-AUCB-containing gel significantly increased thermal hyperemia compared to the vehicle control gel (FIG. 6B).

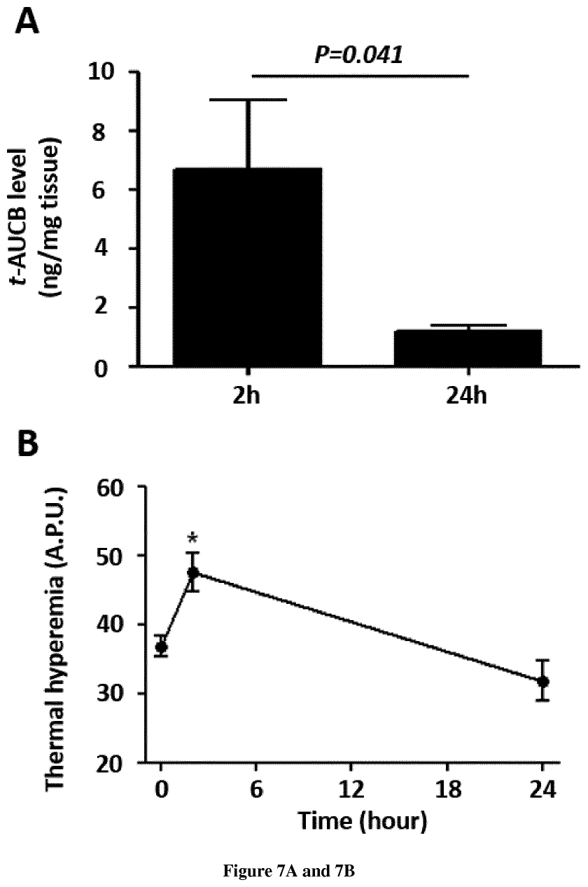

After the 2-hour gel application, t-AUCB was detectable in skin biopsies and skin levels drastically decreased 24 hours after application, demonstrating transdermal permeation of t-AUCB across db/db mouse skin (FIG. 7A). Consistently, thermal hyperemia returned to baseline values 24 hours after application (FIG. 7B).

Plasma quantification showed no systemic diffusion of t-AUCB, assessed 2 and 24 hours after application of the t-AUCB-containing gel, except for one animal (Table 1). In addition, no significant inflammatory infiltrate was observed in mouse skin 2 and 24 hours after gel application (Data not shown).

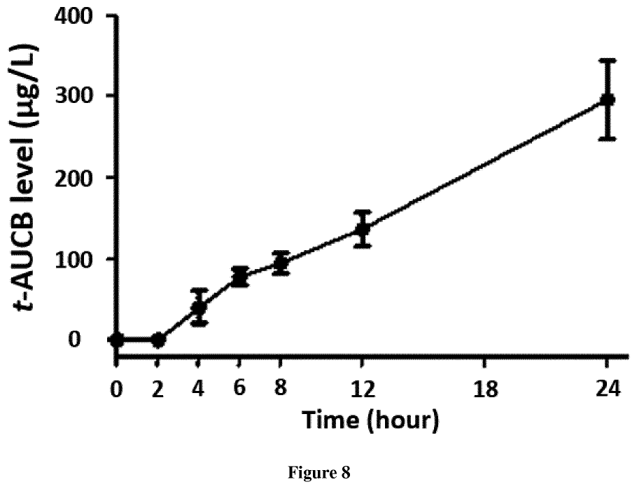

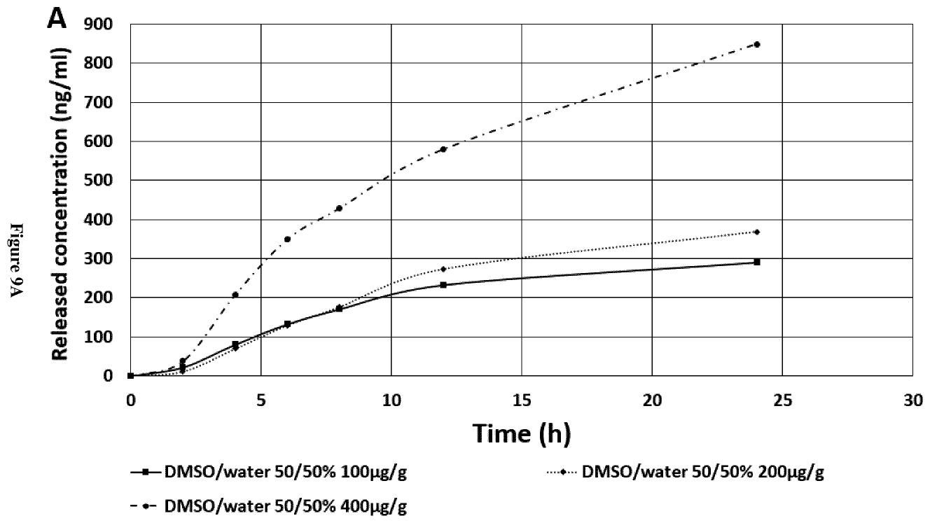

Finally, we observed a continuous diffusion of t-AUCB across pig ear skin from 2 hours after application to over a period of 24 h (FIG. 8).

TABLE 1
Plasma quantification of t-AUCB

| | 2 hours | 24 hours |
|---|---|---|
| Below LOQ (2.4 nM) | 5 (83%) | 4 (100%) |
| Above LOQ (2.4 nM) | 1 (17%) | 0 (0%) |

Data are n (%).
LOQ: limit of quantification.

### Example 5: GSK2256294 Topical Formulations

Method:

First GSK2256294 topical formulations used DMSO and water 50/50% as vehicle. The experiments were conducted with Franz's cell using the skin of a pig's ear. The donor compartment was filled by an 20 μL of solution with a concentration between 100 to 400 μg/g of GSK2256294. The receptor medium was constituted by 4.5 mL of PBS, was at 32° C. and under magnetic stirring. Samples from the receptor compartment were collected at different times over 24 hours to determine the passage flow of GSK2256294. Second GSK2256294 topical formulation using alcohol, isopropanol, propylene glycol and DMSO 50/50% as vehicle with a GSK2256294's concentration at 200 μg/g were tested in the same conditions. Samples were then frozen at −20° C. The t-AUCB quantification was realized by HPLC/MS/MSS.

Results:

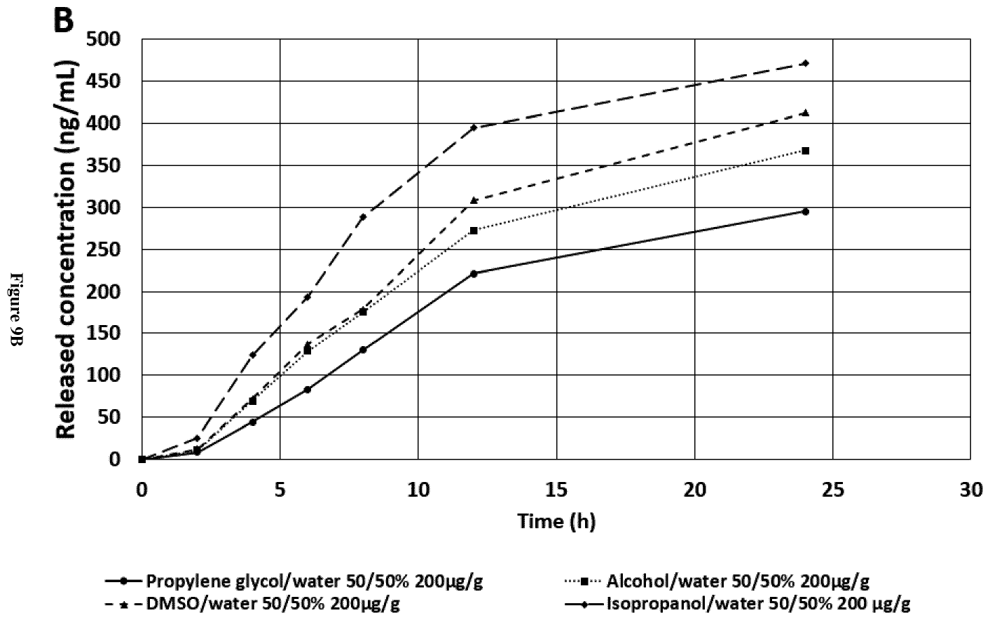

FIG. 9A represents the passage of DMSO/water 50/50% solutions of GSK2256294 with a concentration between 100 and 400 μg/g. The percutaneous flow is higher with a larger concentration of GSK2256294. The flow between 4 and 12 h increases from 41.92 to 102.26 ng/h/cm². The flow through the pigskin was effective from 4 h to 12 h for lower concentrations and from 4 h to 24 h for largest concentration.

FIG. 9B represents the passage of DMSO, Isopropanol, Alcohol or Propylene Glycol water solutions (50/50%) of GSK2256294 with a concentration at 200 μg/g. The percutaneous flows are similar. The flow between 4 and 12 h with propylene glycol is the lowest with 48.49 ng/h/cm², with alcohol 55.9 ng/h/cm², with isopropanol 74.38 ng/h/cm² and with DMSO 64.83 ng/h/cm². The flow through the pigskin was effective from 4 h to 12 h and with a passage rate at 24 h between 32 and 52%.

Discussion:

The major finding of the present study is that sEH inhibition through a topical formulation increases thermal hyperemia, an index of endothelium-dependent microvascular reactivity, in a murine model of diabetes. Because microvascular endothelial dysfunction is a hallmark of the disease, and considering the involvement of impaired cutaneous microcirculation in poor wound healing in diabetes, such strategy could be a relevant therapeutic approach for diabetic foot ulcers (DFUs).

Endothelium-derived epoxyeicosatrienoic acids (EETs) are endothelium-derived vasodilating factors with powerful anti-inflammatory and pro-angiogenic properties that could be useful in the treatment of the cardiovascular complications of type 2 diabetes (6,7). Despite increasing evidence suggesting a possible role for EETs in diabetes-related endothelial dysfunction, no study had previously focused on diabetic skin microvascular dysfunction. The use of thermal hyperemia as a reactivity test in the present work was motivated by the involvement of EETs together with NO in the response to local heating in humans (16).

We observed a reduction in basal skin blood flow in diabetic db/db mice compared to wild-type mice that is probably mainly related to decreased vascular density (17). In addition, although no data were available in animal models of diabetes when we designed the study, we demonstrated an altered microvascular reactivity to thermal hyperemia in diabetic mice. Thus, as shown in humans (18), measuring blood flow response to a standardized local heat stimulus represents an adequate model to study the skin microvascular dysfunction associated with diabetes in mice.

In this context, we tested the impact of a topical formulation containing t-AUCB, an inhibitor of EET degradation by sEH (11,13), on the skin microvascular dysfunction of db/db mice. Quantification of t-AUCB in skin biopsies revealed a significant transdermal permeation of the drug 2 hours after gel application, associated with increased basal skin blood flow and thermal hyperemia. Yet, when comparing the vehicle control gel, we noticed that in fact both formulations similarly increased basal skin blood flow. This result supports previous data showing a direct vasodilating effect of the vehicle DMSO (19). In fact, topical administration of DMSO was even proposed in humans to treat the skin complications of systemic scleroderma, which is also characterized by microvascular dysfunction and a risk of ulcer, but the results from randomized controlled trials were disappointing (20,21). However, DMSO had no effect on reactivity. In contrast, the t-AUCB-containing gel improved thermal hyperemia compared to the vehicle control gel, demonstrating the improvement in skin microvascular reactivity. This result shows that, as previously demonstrated in coronary and peripheral arteries (11,12), sEH plays a major role in the vascular dysfunction associated with type 2 diabetes at the level of the skin. Although our objective in this preliminary study was not to assess the effect of sEH inhibition on wound healing, it provides a first proof-of-principle in an animal model with delayed wound healing (22).

Importantly for potential human use, histological analysis revealed no signs of skin toxicity with the t-AUCB-containing gel. In addition, plasma quantification of t-AUCB in exposed animals showed a limited systemic diffusion of the drug, in only one animal. This may be important because, although first results obtained in the first phases of clinical development suggest that sEH inhibitors were safe (23,24), some data show that increasing EET bioavailability may be associated with adverse effects and in particular may potentiate tumor development (6,7,25,26). Moreover, because mouse skin is thin and shaving of the animals for the experiments lead to an underestimation of the time needed for the transdermal passage of t-AUCB compared to humans, we performed a pharmacokinetic study on isolated human pig ear skin. We observed a progressive and continuous diffusion of t-AUCB that could be particularly useful to prevent and/or treat the skin complications of patients with type 2 diabetes.

In conclusion, our results show that the topical administration of a sEH inhibitor improves skin microvascular reactivity in a model of type 2 diabetes. The absence of skin toxicity, the limited systemic diffusion and the demonstration of a progressive passage of the sEH inhibitor across a skin closed to human support the use of this therapeutic strategy in patients with type 2 diabetes with the expected results of preventing skin complications and in particular DFUs development.

### REFERENCES

Throughout this application, various references describe the state of the art to which this invention pertains. The disclosures of these references are hereby incorporated by reference into the present disclosure.
1. Moxey P W, Hofman D, Hinchliffe R J, Jones K, Thompson M M, Holt P J E. Epidemiological study of lower limb amputation in England between 2003 and 2008. Br J Surg. 2010; 97:1348-1353.
2. Dinh T, Tecilazich F, Kafanas A, Doupis J, Gnardellis C, Leal E, Tellechea A, Pradhan L, Lyons T E, Giurini J M, Veves A. Mechanisms involved in the development and healing of diabetic foot ulceration. Diabetes. 2012; 61:2937-2947.
3. Chao C Y, Cheing G L. Microvascular dysfunction in diabetic foot disease and ulceration. Diabetes Metab Res Rev. 2009; 25:604-614.
4. Jhamb S, Vangaveti V N, Malabu U H. Genetic and molecular basis of diabetic foot ulcers: Clinical review. J Tissue Viability. 2016; 25:229-236.
5. Valacchi G, Zanardi I, Sticozzi C, Bocci V, Travagli V. Emerging topics in cutaneous wound repair. Ann N Y Acad Sci. 2012; 1259:136-144.
6. Lorthioir A, Guerrot D, Joannides R, Bellien J. Diabetic cardiovascular disease—Soluble epoxide hydrolase as a target. Cardiovasc Hematol Agents Med Chem. 2012; 10:212-222.
7. Bellien J, Joannides R, Richard V, Thuillez C. Modulation of cytochrome-derived epoxyeicosatrienoic acids pathway: a promising pharmacological approach to prevent endothelial dysfunction in cardiovascular diseases? Pharmacol Ther. 2011; 131:1-17.
8. Zhao H, Chen J, Chai J, Zhang Y, Yu C, Pan Z, Gao P, Zong C, Guan Q, Fu Y, Liu Y. Cytochrome P450 (CYP) epoxygenases as potential targets in the management of impaired diabetic wound healing. Lab Invest. 2017 July; 97:782-791.
9. Sander A L, Jakob H, Sommer K, Sadler C, Fleming I, Marzi I, Frank J. Cytochrome P450-derived epoxyeicosatrienoic acids accelerate wound epithelialization and neovascularization in the hairless mouse ear wound model. Langenbecks Arch Surg. 2011; 396:1245-1253.
10. Sander A L, Sommer K, Neumayer T, Fleming I, Marzi I, Barker J H, Frank J, Jakob H. Soluble epoxide hydrolase disruption as therapeutic target for wound healing. J Surg Res. 2013; 182:362-367.
11. Roche C, Besnier M, Cassel R, Harouki N, Coquerel D, Guerrot D, Nicol L, Loizon E, Morisseau C, Remy-Jouet I, Mulder P, Ouvrard-Pascaud A, Madec A M, Richard V, Bellien J. Soluble epoxide hydrolase inhibition improves coronary endothelial function and prevents the development of cardiac alterations in obese insulin-resistant mice. Am J Physiol Heart Circ Physiol. 2015; 308:H1020-H1029.
12. Zhang L N, Vincelette J, Chen D, Gless R D, Anandan S K, Rubanyi G M, Webb H K, MacIntyre D E, Wang Y X. Inhibition of soluble epoxide hydrolase attenuates endothelial dysfunction in animal models of diabetes, obesity and hypertension. Eur J Pharmacol. 2011; 654:68-74.
13. Liu J Y, Tsai H J, Hwang S H, Jones P D, Morisseau C, Hammock B D.

Pharmacokinetic optimization of four soluble epoxide hydrolase inhibitors for use in a murine model of inflammation. Br J Pharmacol. 2009; 156:284-296.
14. Roustit M, Cracowski J L. Assessment of endothelial and neurovascular function in human skin microcirculation. Trends Pharmacol Sci. 2013; 34:373-384.
15. Herkenne C, Naik A, Kalia Y N, Hadgraft J, Guy R H. Pig ear skin ex vivo as a model for in vivo dermatopharmacokinetic studies in man. Pharm Res. 2006; 23:1850-1856.
16. Brunt V E, Minson C T. KCa channels and epoxyeicosatrienoic acids: major contributors to thermal hyperaemia in human skin. J Physiol. 2012; 590:3523-3534.
17. Schaefer C, Biermann T, Schroeder M, Fuhrhop I, Niemeier A, Riither W, Algenstaedt P, Hansen-Algenstaedt N. Early microvascular complications of prediabetes in mice with impaired glucose tolerance and dyslipidemia. Acta Diabetol. 2010; 47:19-27.
18. Fuchs D, Dupon P P, Schaap L A, Draijer R. The association between diabetes and dermal microvascular dysfunction non-invasively assessed by laser Doppler with local thermal hyperemia: a systematic review with meta-analysis. Cardiovasc Diabetol. 2017; 16:11.
19. Kaneda T, Sasaki N, Urakawa N, Shimizu K. Endothelium-dependent and -independent vasodilator effects of dimethyl sulfoxide in rat aorta. Pharmacology. 2016; 97:171-176.
20. Scherbel A L. The effect of percutaneous dimethyl sulfoxide on cutaneous manifestations of systemic sclerosis. Ann N Y Acad Sci. 1983; 411:120-30.
21. Williams H J, Furst D E, Dahl S L, Steen V D, Marks C, Alpert E J, Henderson A M, Samuelson C O Jr, Dreyfus J N, Weinstein A, et al. Double-blind, multicenter controlled trial comparing topical dimethyl sulfoxide and normal saline for treatment of hand ulcers in patients with systemic sclerosis. Arthritis Rheum. 1985; 28:308-314.
22. Sullivan S R, Underwood R A, Gibran N S, Sigle R O, Usui M L, Carter W G, Olerud J E. Validation of a model for the study of multiple wounds in the diabetic mouse (db/db). Plast Reconstr Surg. 2004; 113:953-960.
23. Chen D, Whitcomb R, MacIntyre E, Tran V, Do Z N, Sabry J, Patel D V, Anandan S K, Gless R, Webb H K. Pharmacokinetics and pharmacodynamics of AR9281, an inhibitor of soluble epoxide hydrolase, in single- and multiple-dose studies in healthy human subjects. J Clin Pharmacol. 2012; 52:319-328.
24. Lazaar A L, Yang L, Boardley R L, Goyal N S, Robertson J, Baldwin S J, Newby D E, Wilkinson I B, Tal-Singer R, Mayer R J, Cheriyan J. Pharmacokinetics, pharmacodynamics and adverse event profile of GSK2256294, a novel soluble epoxide hydrolase inhibitor. Br J Clin Pharmacol. 2016; 81:971-979.
25. Panigrahy D, Edin M L, Lee C R, Huang S, Bielenberg D R, Butterfield C E, Barnés C M, Mammoto A, Mammoto T, Luria A, Benny O, Chaponis D M, Dudley A C, Greene E R, Vergilio J A, Pietramaggiori G, Scherer-Pietramaggiori S S, Short S M, Seth M, Lih F B, Tomer K B, Yang J, Schwendener R A, Hammock B D, Falck J R, Manthati V L, Ingber D E, Kaipainen A, D'Amore P A, Kieran M W, Zeldin D C. Epoxyeicosanoids stimulate multiorgan metastasis and tumor dormancy escape in mice. J Clin Invest. 2012; 122:178-191.
26. Sausville L N, Gangadhariah M, Chiusa M, Mei S, Wei S, Zent R, Luther J M, Shuey M M, Capdevila J H, Falck J R, Guengerich F P, Williams S M, Pozzi A. The cytochrome P450 slow metabolizers CYP2C9*2 and CYP2C9*3 directly regulate tumorigenesis via reduced epoxyeicosatrienoic acid production. Cancer Res. 2018 Jul. 16. pii: canres.3977.2017.

1. A method of treating skin microvascular dysfunction in a subject in need thereof comprising topically administering to the subject a therapeutically effective amount of a sEH inhibitor.

2. The method of claim 1 wherein the subject suffers from diabetes mellitus.

3. The method of claim 1 wherein the subject suffers from type 2 diabetes.

4. The method of claim 1 wherein the subject suffers from systemic sclerosis (SSc).

5. The method of claim 1 wherein the subject suffers from a disease or condition selected from the group consisting of inherited or recessive myopathies, muscle-wasting diseases, conditions of muscle atrophy or attenuation, protracted disuse, weakness induced by surgery, drug-induced myopathy and rhabdomyo lysis.

6. The method of claim 1, wherein the skin microvascular dysfunction is a diabetic ulcer.

7. The method of claim 1 wherein the inhibitor of sEH is selected from the group consisting of 3-(4-chlorophenyl)-1-(3,4-dichlorphenyl)urea or 3,4,4′-trichlorocarbanilide (TCC); 12-(3-adamantan-1-yl-ureido) dodecanoic acid (AUDA); 1-adamantanyl-3-(5-[2-(2-ethoxyethoxy)ethoxy]pentyl]}urea (AEPU); 1-(1-acetypiperidin-4-yl)-3-adamantanylurea (APAU); trans-4-[4-(3-Adamantan-1-yl-ureido)-cyclohexyloxy]-benzoic acid (tAUCB); cis-4-[4-(3-Adamantan-1-yl-ureido)-cyclohexyloxyj-benzoic acid (cAUCB); 1-(1-methylsulfonyl-piperidin-4-yl)-3-(4-trifluoromethoxy-phenyl)-urea (TUPS); trans-4-{4-[3-(4-Trifluoromethoxy-phenyl)-ureido]-cyclohexyloxy}-benzoic acid (tTUCB); 1-trifluoromethoxyphenyl-3-(1-propionylpiperidin-4-yl) urea (TPPU); 1-(1-ethylsulfonyl-piperidin-4-yl)-3-(4-trifluoromethoxy-phenyl)-urea (TUPSE) 1-(1-(cyclopropanecarbonyl)piperidin-4-yl)-3-(4-(trifluoromethoxy)phenyl)urea (CPTU); trans-N-methyl-4-[4-(3-Adamantan-1-yl-ureido)-cyclohexyloxy]-benzamide (tMAUCB;) trans-N-methyl-4-[4-((3-trifluoromethyl-4-chlorophenyl)-ureido)-cyclohexyloxy]-benzamide (tMTCUCB); cis-N-methyl-4-{4-[3-(4-trifluoromethoxy-phenyl)-ureido]-cyclohexyloxy}-benzamide (cMTUCB); and 1-cycloheptyl-3-(3-(1,5-diphenyl-1H-pyrazol-3-yl)propyl)urea (HDP3U).

8. The method of claim 1 wherein the sEH inhibitor is GSK2256294.

9. The method of claim 1 wherein the sEH inhibitor is tAUCB.

10. The method of claim 1 wherein the sEH inhibitor is formulated as a gel, a solution, a suspension, a cream or a patch.

11. The method of claim 6 wherein
the inherited or recessive myopathy is a muscular dystrophy; and/or
the muscle-wasting disease is cachexia; and/or
the condition of muscle atrophy or attenuation is sarcopenia; and/or
the protracted disuse is due to paralysis, coma, extended bed rest, and/or an Intensive Care Unit (ICU) stay); and/or
the surgery is joint replacement surgery.

12. The method of claim 11, wherein the cachexia is due to an underlying illness selected from the group consisting of acquired immunodeficiency diseases [AIDS], rheumatoid arthritis, cancer, chronic obstructive pulmonary disease [COPD], and cirrhosis.

13. The method of claim 11, wherein the sarcopenia is due to aging.

14. The method of claim 6, wherein the diabetic ulcer is a foot diabetic ulcer.
